(19) United States
(12) Patent Application Publication
(10) Pub. No.: US 2023/0200826 A1
(43) Pub. Date: Jun. 29, 2023

(54) A SURGICAL METHOD

(71) Applicant: Orthopaedic Innovations Pty Ltd, Ipswich, Queensland (AU)

(72) Inventor: Michael Mcauliffe, Ipswich, Queensland (AU)

(21) Appl. No.: 17/927,258

(22) PCT Filed: May 25, 2021
(86) PCT No.: PCT/AU2021/050492

§ 371 Date: Nov. 22, 2022

(30) Foreign Application Priority Data
May 25, 2020 (AU) 2020901686

(51) Int. Cl. A61B 17/15; A61B 34/10

(52) U.S. Cl. CPC A61B 17/155; A61B 34/10; A61B 2034/108; A61B 2034/105

(57) ABSTRACT

The present disclosure is directed to a method of surgery, such as knee replacement surgery and hip replacement surgery that achieves optimal implant sizing and placement within a surgically repaired joint.

### FIELD

THIS DISCLOSURE relates generally to a method of performing surgery. In particular, the present disclosure is directed to a method of surgery, such as knee replacement surgery and hip replacement surgery that achieves optimal implant sizing and placement within a surgically repaired joint.

### BACKGROUND

Currently there is a significant focus on the use of robotics within joint replacement surgery. This is based largely upon the premise that robots will be able to help gather information that can be used for more precise surgical planning and that they will more precisely deliver this surgical plan. This premise is valid if there is good evidence that such precision has a high correlation to clinical outcomes. The current data for this proposition, however, is limited. Furthermore, the various robotic systems carry significant costs particularly in terms of hardware capital expenditure, software updates, elevated staffing requirements and increased theatre times both during surgery and between case turnover times. All health systems are under financial pressure and it is vital to demonstrate cost-effectiveness of technological adjuncts to joint replacement surgery.

Preoperative education is increasingly employed to improve surgical outcomes. Structured programs which have been developed particularly in recent times to enable “day case” joint replacement surgery have shown improved rates of surgical complications and patient reported outcomes. Despite these efforts it is not uncommon for patients to make statements in the post-operative setting demonstrating that they still had a limited understanding of the underlying pathology and the overall surgical process. This is unsurprising given the amount of information that is supplied to patients in the perioperative period. Whilst they might understand the general nature of the problem, patient's regularly express a post-operative desire to understand exactly the pathology within their own osteoarthritic (or other disease damaged) joint.

Accordingly, there remains a need for improved methods of joint surgery that are cost effective and improve patient outcomes.

### SUMMARY

The present disclosure is broadly directed to a method of performing joint surgery and, in particular, joint replacement surgery, such as hip or knee replacement surgery. The method may be performed to determine the optimal implant size to be implanted and/or whether additional bony resections are required prior to implant insertion by way of coupling models of resected bone portions and the patient's joint prior to surgery. The present disclosure is further directed to methods of demonstrating the severity of a disease, disorder or condition in a patient's joint, such as osteoarthritis, to the patient.

In a first aspect, the present disclosure provides a method of performing surgery on a joint or bone of a patient, including the steps of:

(a) comparing a model of one or more resected portions resected from the joint or bone with a model of one or more implants and/or a model of the patient's joint or bone prior to surgery; and

(b) determining a course of action associated with surgery on the joint or bone based at least partly on the comparison in (a).

In a second aspect, the present disclosure relates to a method of determining or selecting an implant for implantation into a patient's joint or bone, said method including the steps of:

(a) comparing a model of one or more resected portions resected from the joint or bone with a model of one or more implants and/or a model of the joint or bone prior to surgery; and

(b) determining or selecting the implant for implantation into a patient's joint or bone based at least partly on the comparison in (a).

The method of the second aspect suitably includes the further step of implanting the implant determined in step (b) into the patient's joint or bone.

In certain embodiments, the above methods include comparing the model of one or more resected portions with the model of one or more implants and the model of the patient's joint or bone prior to surgery.

In some embodiments, the above methods include comparing the model of one or more resected portions with the model of one or more implants.

In other embodiments, the above methods include comparing the model of one or more resected portions with the model of the patient's joint or bone prior to surgery.

In some embodiments of the first and second aspects, the model of the one or more resected portions, the model of the one or more implants and/or the model of the patient's joint or bone are or comprise respective three dimensional (3D) models.

Suitably, the method of the aforementioned two aspects further includes the initial step of generating the model of the one or more resected portions resected from the joint or the bone. By way of example, the model of the one or more resected portions resected from the joint or the bone can be generated at least in part by a scanning device.

In some embodiments, the scanning device is or comprises a laser scanner, an ultrasound scanner, an x-ray device and/or an infrared scanner.

Referring to the first and second aspects, the method suitably further includes the initial step of generating the model of the patient's joint or bone prior to surgery. In various embodiments, the model of the patient's joint or bone is generated at least in part by radiological imaging (such as plain radiographs and EOS medical imaging), magnetic resonance imaging (MRI) and/or computed tomography (CT). In particular embodiments, the model of the patient's joint or bone prior to surgery is a 3D model.

For the above aspects, the method may further include the initial step of generating the model of the one or more implants prior to surgery.

The above methods may include the further step of generating a model of residual bone. To this end, step (a) suitably comprises overlaying the model of the one or more resected portions resected from the joint or bone with the model of the patient's joint or bone. In this regard, the method may further comprise subtracting the model of the one or more resected portions resected from the joint or bone from the model of the patient's joint or bone to generate the model of residual bone thereof. In some embodiments, the method includes the step of comparing the model of the one or more implants and/or the model of the patient's joint or bone with the model of residual bone.

In alternative embodiments, the model of residual bone is generated at least in part by a scanning device, such as a hand held scanning device.

For the method of the first aspect, the course of action suitably comprises further resection of one or more surfaces or bones of the patient's joint or bone, such as one or more resected surfaces or bones of the joint or bone. Additionally, the method of the second aspect, may include further resection of one or more surfaces or bones of the joint or bone, such as one or more resected surfaces or bones of the joint or bone. To this end, further resection can be the course of action when the comparison of: (i) the model of the one or more resected portions and/or the model of residual bone with the model of the patient's joint or bone; and/or (ii) the model of the one or more implants with the model of the one or more resected portions and/or the model of residual bone; indicates insufficient and/or improper resection of one or more bones of the joint or the bone. In some embodiments, the present methods include further resection of one or more of a resected distal femoral surface, a resected posterior femoral surface, a resected anterior femoral surface, a resected proximal tibial surface and a resected proximal femoral surface. Suitably, further resection of the one or more resected surfaces or bones of the joint or bone is based at least partly on the comparison in (i) and/or (ii). Accordingly, the methods of the above aspects may further include the step of determining one or more further resection planes, such as one or more further resection planes of a resected distal femoral surface, a resected posterior femoral surface, a resected anterior femoral surface, a resected proximal tibial surface and/or a resected proximal femoral surface.

With respect to the first and second aspects, the method suitably includes determining an implant size for the patient's joint or bone based on the comparison in (a). In some embodiments, the step of determining the implant size for the patient's joint includes determining one or more of a tibial insert size, a tibial implant size, a distal femoral implant size, a proximal femoral implant size and a pelvic implant size. Accordingly, the implant can be selected from the group consisting of a tibial insert, a tibial implant, a distal femoral implant, a proximal femoral implant, a pelvic implant and any combination thereof. The method suitably includes the further step of implanting into the patient's joint or bone an implant that corresponds or correlates to the determined implant size.

In some embodiments, the method of the first and second aspects can further include determining a position or orientation of the implant in relation to the patient's joint or bone based on the comparison in (a).

Suitably, the method of the first and second aspects further includes the initial step of resecting the one or more resected portions from the joint or bone.

In particular embodiments, the method of the above aspects further includes the initial step of making a preliminary resection in the joint or bone so as to produce the one or more resected portions. Referring to the method of the first aspect, the course of action can comprise; (i) determining one or more further resection planes for the joint or bone based at least partly on the preliminary resection; and (ii) optionally further resecting the joint or bone based at least partly on the one or more further resection planes. Referring to the second aspect, the method may further include the steps of: (i) determining one or more further resection planes for the joint or bone based at least partly on the selected implant and/or the preliminary resection; and (ii) optionally further resecting the joint or bone based at least partly on the one or more further resection planes.

In some embodiments, the aforementioned methods include the further steps of measuring a soft tissue tension of the joint and/or utilising the soft tissue tension measure to determine at least partly the course of action associated with surgery on the joint. By way of example, the soft tissue tension and/or the model of the resected portions may facilitate determining a total resection gap in the model of the patient's joint or bone or the model of residual bone.

In a third aspect, the present disclosure provides a method of performing surgery on a joint or bone of a patient, including the steps of:

(a) comparing a model of one or more resected portions resected from the joint or bone by a preliminary resection with a model of the patient's joint or bone prior to surgery; and

(b) determining a course of action associated with surgery on the joint or bone based at least partly on the comparison in (a).

Suitably, the present method includes one or more of those features and/or steps provided for the methods of the first and second aspects.

With respect to step (a), this may include generating a model of residual bone of the patient's joint or bone.

In particular embodiments, the course of action can include determining one or more further resection planes for the joint or bone. For such embodiments, the method may further include the step of further resecting the joint or bone based at least partly on the one or more further resection planes.

In some embodiments, the course of action can include determining a positioning of one or more cutting guides on the joint or bone to make one or more further resections therein. For such embodiments, the method may further include the step of further resecting the joint or bone based at least partly on the positioning of the one or more cutting guides thereon.

Suitably, the method further includes the step of generating a model of one or more further resected portions that are produced or generated by the further resections in the patient's joint or bone.

In some embodiments, the present method further includes the steps of:

(c) comparing the model of the one or more further resected portions with a model of one or more implants, the model of the patient's joint or bone prior to surgery and/or the model of residual bone; and

(d) determining or selecting an implant for implantation into a patient's joint or bone based at least partly on the comparison in (c).

Suitably, step (c) of the present method includes the further step of generating a further model of residual bone based on such a comparison. In some embodiments, step (c) further includes comparing the further model of residual bone with the model of one or more implants.

In a fourth aspect, the invention resides in a method of demonstrating the severity of a disease, disorder or condition in a patient's joint to the patient, said method including the steps of:

(a) generating a model of one or more resected portions resected from the joint; and

(b) providing the model to the patient.

Suitably, the model is or comprises a 3D model.

Suitably, the disease, disorder or condition is or comprises arthritis, such as osteoarthritis or rheumatoid arthritis.

In particular embodiments, the model of the one or more resected portions is generated at least in part by a scanning device. In this regard, the scanning device may be or comprise a laser scanner, an ultrasound scanner, an x-ray device and/or an infrared scanner.

In a fifth aspect, the invention provides a system for assisting a surgeon in performing surgery on a joint of a patient, the system comprising a processor configured for:

(a) comparing a model of one or more resected portions resected from the joint with a model of one or more implants and/or a model of the patient's joint prior to surgery; and

(b) optionally determining a course of action associated with surgery on the patient's joint based at least partly on the comparison in (a).

In a sixth aspect, the present disclosure provides an apparatus or system for determining or selecting an implant for implantation into a patient's joint or bone, the apparatus or system comprising a processor configured for:

(a) comparing a model of one or more resected portions resected from the joint or bone with a model of one or more implants and/or a model of the joint or bone prior to surgery; and

(b) optionally determining or selecting the implant for implantation into a patient's joint or bone based at least partly on the comparison in (a).

In some embodiments of the two aforementioned aspects, the processor may further utilise a measure of soft tissue tension of the joint to determine at least partly the course of action or the implant for implantation.

Suitably, the system of the above two aspects further comprises a scanning device for generating the model of the one or more resected portions resected from the joint.

Suitably, the system of the fifth and sixth aspects is for use in the method of the first, second and third aspects.

In a seventh aspect, the invention relates to a computer-readable medium having stored thereon a computer program, which, when executed by a computer, causes the computer to perform the method of the first, second and third aspects.

Referring to the aforementioned aspects, the joint is suitably a knee joint, a shoulder joint, an ankle joint or a hip joint.

For the above aspects, the patient is suitably a mammal and more preferably a human.

It will be appreciated that the indefinite articles “a” and “an” are not to be read as singular indefinite articles or as otherwise excluding more than one or more than a single subject to which the indefinite article refers.

As used herein, unless the context requires otherwise, the words “comprise”, “comprises” and “comprising” will be understood to mean the inclusion of a stated integer or group of integers but not the exclusion of any other non-stated integer or group of integers.

### BRIEF DESCRIPTION OF THE DRAWINGS

In order that the present invention may be readily understood and put into practical effect, reference will now be made to the accompanying illustrations, wherein like reference numerals are used to refer to like elements.

FIG. 1: is a flow chart of how an embodiment of the method of the invention may proceed.

FIG. 2: illustrates an apparatus or system according to one embodiment of the invention.

### DETAILED DESCRIPTION

The present invention relates to a method ofjoint surgery and, in particular, joint replacement surgery, that includes comparing respective models of bony resections and joint anatomy prior to surgery so as to develop a model of residual bone. The models of resected and/or residual bone may then be used to assist a surgeon in determining optimal implant sizing and positioning, the accurate planning of cutting guide positioning and whether further resection of the joint is required. The invention is further directed to methods of demonstrating the severity of a disease, disorder or condition in a patient's joint to the patient.

While the methods described herein are particularly suited for use in total knee replacement or arthroplasty (TKR/TKA), the present invention has general applicability to all types of joints (e.g., hips, ankles, elbows, shoulders, wrists and fingers) and replacement surgery thereof that requires accurate gap balancing, joint alignment and implant sizing.

While the principles described herein are based on methods of surgery for humans, this invention may also be extended to other mammals such as livestock (e.g.

cattle, sheep), performance animals (e.g. racehorses) and domestic pets (e.g. dogs, cats), although without limitation thereto.

It will be appreciated current navigation or robotics systems typically utilise 3-dimensional (3D) models of bones which are developed from preoperative images or from registration processes during surgery. Bone is then resected from the joint in question as required, and the amount of remaining bone is assessed. This places primary importance on the bone that is retained rather than the bone that is removed or resected. Reasons for this include that it can be difficult to quantify the amount of bone removed during small recuts and the amount of bone that is destroyed by the saw blade itself.

Approximately one third of knee replacements in Australia utilise robotics or computer navigation. Australia is an outlier in terms of the use of this technology. Most countries utilise robotics/navigation at a less than 10% rate for knee replacement. The rates of navigation in hip replacement surgery are significantly lower for all jurisdictions. Therefore, the majority of joint replacement surgery that occurs around the world is undertaken with conventional instrumentation. This relies upon individual surgeon technique and experience to achieve a good surgical outcome. These procedures, however, lack objective feedback for the surgeon. This is important as there is clear evidence that surgeons are unable to appreciate small angular and bone thickness differences that occur during surgery. Up to 20% of patients having knee replacements also report some degree of dissatisfaction with the outcome and such technical inadequacies that occur during surgery are likely to contribute to a large proportion of this dissatisfaction. Advantageously, the method described herein provides a cost-effective means of providing objective feedback to surgeons that may be of value in improving patient outcomes.

Accordingly, in one aspect the invention provides, in part, a method of performing surgery on a joint of a patient, including the steps of:

(a) comparing a model of one or more resected portions resected from the joint with a model of one or more implants and/or a model of the joint prior to surgery; and

(b) determining a course of action associated with surgery on the joint based at least partly on the comparison in (a).

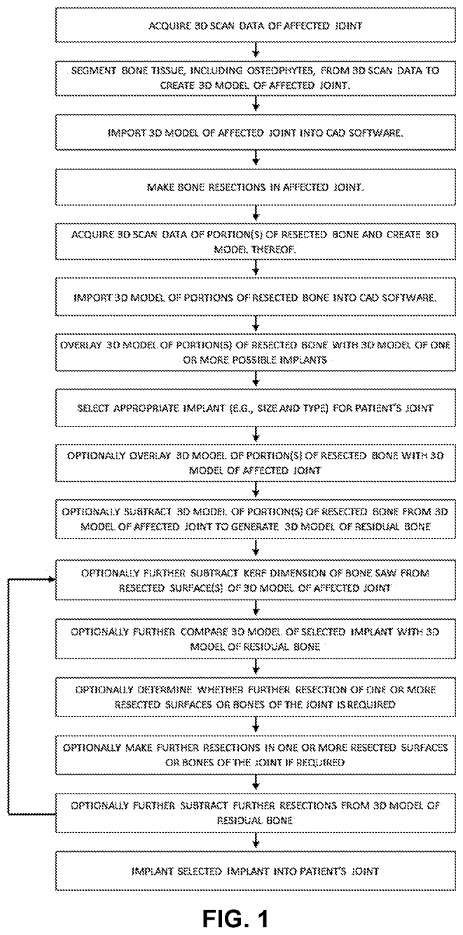

FIG. 1 demonstrates an embodiment of a method of performing joint surgery according to the present disclosure.

Based on this, the present method may include the initial step of generating a model of the patient's joint prior to surgery. Suitably, patient-specific anatomical data is obtained pre-operatively using one or more non-invasive imaging modalities, such as radiological imaging, including plain radiographs and EOS medical imaging systems that can be used for the 2D or 3D visualisation of bones and joints, computerised tomography (CT)/computerised axial tomography (CAT), magnetic resonance imaging (MRI), inclusive of full limb MRI, ultrasound and/or other conventional means. The patient-specific anatomical data obtained therefrom may then be pre-processed and/or converted to form a patient-specific model of the joint and the bony structures thereof Such a patient-specific model may include a two dimensional model (e.g., radiographs, 2D slices of MRI) and/or a three dimensional model. Preferably, the patient-specific model is or comprises a three dimensional model, such as a three-dimensional (3D) computer aided design (CAD) model. Generally, segmentation of the bone tissue, including osteophytes, from the patient-specific anatomical data is performed to thereby create a three dimensional model of the affected joint and the resected portions of bone.

Surgery of the patient's joint may then proceed as per standard or conventional methods known in the art. By way of example, conventional cutting guides can be placed in a position determined to be optimal by the surgeon. Initial resections can then be undertaken, such as to a distal femoral portion (e.g., a distal femoral resection), a posterior femoral portion (e.g., a posterior femoral resection, a posterior chamfer resection), an anterior femoral portion (e.g., an anterior femoral resection, an anterior chamfer resection), a proximal tibial portion, a proximal femoral portion and a pelvic portion, with an emphasis on optimal saw technique.

It is envisaged that the present method may also be utilised with customised or patient specific cutting guides or image derived instrumentation (IDI). These cutting guides are now used in >10% of knee replacement surgery in Australia and the United States. Such cutting guides typically come with a surgical plan that includes the amount of bone that should be resected. Currently, the amount of resected bone is determined by visual inspection or using callipers to measure a known point on the resected bone. Such means for assessing the amount of bone resected from a joint, however, can be inaccurate. By way of example, calliper measurement of resected bone only measures bone thickness at a single point. Even if this point is accurate it can fail to assess the overall resection and therefore whether the surgical plan is being achieved. Additionally, and whilst these surgical guides are being increasingly used, there is no current evidence that they are decreasing surgical revision rates or improving surgical outcomes (McAuliffe et al., J Bone joint Surg Am 2019 Apr. 3; 10 (7) 580-588). They do, however, offer heightened efficiency within theatre and are potentially more cost effective than navigation/robotic technologies. Nonetheless, it will be appreciated that the current method of the present disclosure can be utilised as a means of rapidly assessing the accuracy of customised cutting guides as surgery is occurring.

Following resection of the patient's joint, the model, inclusive of a 3D model, of the one or more bone portions resected therefrom may then be generated. In this regard, a model of the one or more resected bone portions may be generated or created by any means or method known in the art. In particular embodiments, the model of the one or more resected bone portions is generated at least in part by a scanning device. Suitably, the scanning device is configured to acquire 3D structural information about the resected bone portion. To this end, the scanning device may include any technology known in the art for digitally acquiring the shape of a 3D object, such as contact, non-contact active (e.g., time-of-flight, triangulation, structured light, modulated light) and non-contact passive (e.g., photogrammetry) technologies and any combination thereof. Suitably, the scanning device contains one or a plurality of sensors, cameras, scanning units or the like for acquiring 3D structural information about the resected bone portions. In certain embodiments, the scanning device includes one or a plurality of laser scanning units, an ultrasound scanner, an x-ray or radiographic device and/or infrared scanning units. With respect to the x-ray device, this may include conversion of two dimensional (2D) radiographic images generated thereby to 3D models, as is known in the art.

Suitably, the scanning device is of dimensions suitable for inclusion in an operating theatre or the like. In some embodiments, the scanning device is a handheld device, such as a mobile phone, tablet or other compact electronic device, configured for generating the model of the one or more bone portions resected from the joint.

In other embodiments, the scanning device includes a housing of suitable dimensions for receiving the resected bone portions therein for scanning. Such a scanning device may also be configured to rotate the resected bone portions and/or the one or plurality of scanning units therein during use so that all parts of the resected bone portions are photographed and/or scanned and consequently a three-dimensional image of all parts of the resected bone portions may be generated. The scanning device of this embodiment may further include a display or be operably connected to a remote display by any wired or wireless means for displaying, for example, the 3D model of the resected bone portions or information derived therefrom. Such a scanning device is suitably made of materials appropriate for multiple uses and hence repeated cleaning and sterilisation thereof.

Referring to FIG. 1, the amount of resected bone can then be directly assessed, such as via appropriate software, which for example may overlay the model of the one or more resected bone portions with one or more models of commercially available surgical implants. This allows for a direct comparison of the amount of bone and cartilage resected compared to the amount of implanted material that will be returned to the patient's joint, simplifying decisions such as which might be most appropriate implant type and size to trial in the patient. In particular embodiments, the implant to be considered or assessed includes a tibial insert, a tibial implant, a distal femoral implant, and a proximal femoral implant (inclusive of stem, neck and head implants). By extension, the step of determining the implant size for the patient's joint can include determining a tibial insert size, a tibial implant size, a distal femoral implant size and/or a proximal femoral implant size for implantation into the patient's joint.

By way of example, the model of the implant may be selected from one or more standard prosthetic devices, or custom prosthetic devices. The model of the implant may be obtained from one or more product lines which may be from one or more implant manufacturers as are known in the art. Said model typically indicates the size and/or positioning of one or more bony resections needed to fit a particular standard or custom prosthetic device. With respect to total knee replacement surgery, the model selected based on the above comparison is suitably of a prosthesis or implant that has been sized and fitted appropriately for best coverage, bone conservation, extension gap stability, mid-flexion gap stability, flexion gap stability, patella tracking and/or placement without anterior femoral notching.

Additional surgical parameters may also be assessed by this comparison of the two models, such as determining tibial baseplate size and whether the tibial implant may be disposed more superiorly or inferiorly relative to the anterior or posterior aspect of the tibia when compared to the native or unresected tibia. Typically, tibial prostheses or implants include a tibial insert, bearing or meniscal replacement component having a concave articular portion configured for articulation with the femoral prosthesis and a tibial implant or tray to which the bearing or meniscal replacement component of the tibial prosthesis may be secured. The tibial tray is generally secured to the bone stock of a resected proximal tibia. As is well known in the art, the bearing or meniscal replacement component is used to provide an appropriate level of friction and contact area at the interface between the femoral component and the tibial bearing component. This assessment will therefore give information around the tibial slope of the resection and the likely balance of the knee as it moves into flexion. These principles could equally be applied to other types of implants, such as the femoral component for knee replacement surgery. To this end, the femoral and tibial resection measurements can be combined for further surgical guidance or planning.

It is envisaged that the present method can also be applied to hip replacement surgery. As will be appreciated by the skilled artisan, hip replacement components typically include an elongated femoral stem component, which is typically metallic and has a lower end to fit endwise into a corresponding recess formed in a femur and a neck portion that extends generally angularly from the upper end of the femoral stem component. An upper end portion of the femoral stem component typically includes a tapered portion, such as a tapered recess/bore or a tapered extension/nose (i.e., a trunnion), adapted to receive a corresponding tapered extension/nose or recess/bore respectively in a femoral head component of the orthopaedic implant. The femoral head component is generally metallic or ceramic and is of suitable dimensions to be received into a socket defined by the joint of interest.

When installed on the femoral stem component, the femoral head component is positioned to bear on either the patient's natural acetabulum or an acetabular component which has been implanted into the patient's pelvis to replace his or her acetabulum. In such a manner, the orthopaedic implant and the natural or artificial acetabulum collectively function as a system which replaces the natural joint of the patient's hip.

Suitably, CAD programs, biomechanical modelling software and Finite Element Analysis software or the like may be utilised to virtually test a selected implant's performance. Software may perform iterative test runs to predict whether or not small adjustments to the positioning of the selected implant are necessary to optimise performance of both the kinematics of the prosthesis/joint and tension/balance of the periarticular soft tissues.

From FIG. 1, the method of the present aspect may further include overlaying the model of the one or more bone portions resected from the joint with the model of the patient's joint. In this regard, the model of the one or more bone portions resected may be subsequently subtracted from the model of the patient's joint prior to surgery to generate a model of residual bone thereof. In alternative embodiments, however, the model of residual bone is generated directly by scanning the resected joint or bone with a scanner, such as with a handheld scanner, as are known in the art.

Additional adjustments to the model of residual bone may be made, such as further comparing or subtracting estimated kerf distances or dimensions (e.g., about 0.25, 0.5, 0.75, 1.0, 1.5, 2.0, 2.5, 3.0 mm etc inclusive of any range therein) of a saw or resection blade from one or more resected surfaces thereof. As will be appreciated, the amount of bone removed by a saw blade corresponds to the thickness of the saw blade and a small increased amount due to the vertical translation thereof (i.e., kerf). By way of example, a saw blade that is 1.27 mm thick is likely to remove approximately 1.5 mm of bone (i.e., have a kerf dimension of approximately 1.5 mm).

This model of residual bone of the patient's joint or bone may then be directly assessed by a surgeon or other qualified user and/or compared with a model of an implant previously determined to be an appropriate shape, size and/or type. Alternatively, the model of residual bone of the patient may be compared in a similar manner to that described herein with a range of implant models of known shape, size and/or type by, for example, software that then determines or selects the implant most appropriate for the patient's joint based on this model of residual bone. Once selected by a user, the model of the implant, and/or one or more dimensions thereof, can then be superimposed onto the model of residual bone. Preferably, the model of the selected implant is positioned on the model of residual bone so as to be appropriately aligned with the required resection planes and optionally allow for appropriate articulation thereof with an adjacent implant. Accordingly, the method described herein can further include determining a position or orientation of the implant in relation to the patient's joint based on the models of resected bone and residual bone. This will allow the chosen implant or prosthesis to mimic the correct native anatomy of the patient as needed and will also allow for corresponding implants (e.g., tibial and femoral implants) to be adjusted in relation to each other if required from analysis of the generated 3D models and other available surgical data.

As will be appreciated and as provided in FIG. 1, the present method can also operate by simply analysing the one or more resected bone portions and comparing a model thereof to the model of the one or more implants, and without generating a model of residual bone. As such, the present method may be based solely on replacing the resected bone and cartilage with an equivalent amount of prosthesis. When used in this manner, the present method may advantageously provide a more precise version of measured resection surgery, which is typically undertaken or estimated with point calliper measurements.

Based on the comparison in step (a), the course of action may comprise further resection of one or more resected surfaces or bones of the joint, particularly in situations in which the model of residual bone and/or the comparison of the model of residual bone with the model of the selected implant indicates insufficient, inadequate, inaccurate and/or improper resection of one or more bones of the joint. Under such circumstances, the method of the present aspect may include further resection of one or more of a resected distal femoral surface, a resected posterior femoral surface, a resected anterior femoral surface, a resected proximal tibial surface, a resected proximal femoral surface and a resected pelvic surface as required.

In this regard, the present method can be utilised to assess bone resections which are typically not reviewed or assessed in great detail by a surgeon during surgery. By way of example, the chamfer cuts or resections of the distal femur (i.e., anterior and posterior chamfer cuts or resections) during knee replacement surgery can be directly compared to the model of the selected implant and hence directly compared to the optimal dimensions of the chamfer resections required. The amount of bone resected from the anterior femur is also highly variable and very important to the balance of the patellofemoral joint. In addition, the shape and position of the native trochlear groove can be related to the shape and position of the prosthetic trochlear groove. This can be important to achieving appropriate patellofemoral forces and tracking in the implanted knee.

By overlaying or comparing the model of the implant selected for implantation into the patient's joint with both the model of residual bone and the model of the resected bone portions, this advantageously provides for two points of assessment as to the suitability of the implant as well as allow detailed surgical decisions to occur in real time. Such a surgical method would also be useful for younger, more demanding patients and those patients presenting with more complex joint deformities. The present method is also significantly simpler and cheaper than current robotics-based systems, as it utilises less disposables (e.g., pins, navigation trackers), does not require intraoperative registration of the patient's joint and requires less hardware and staffing and shorter theatre times.

When the initial resections of the patient's joint are determined to be inaccurate and/or insufficient a recut can occur. In particular embodiments, the present method includes further resection of one or more of a resected distal femoral surface, a resected posterior femoral surface, a resected anterior femoral surface, a resected proximal tibial surface, a resected proximal femoral surface and a resected pelvic surface. As such, the present method may include the step of determining one or more resection planes for the patient's joint or bone. By way of example, this may include one or more of an anterior femoral resection plane, an anterior chamfer resection plane, a distal femoral resection plane, a posterior femoral resection plane, a posterior chamfer resection plane, a proximal femoral resection plane and a pelvic resection plane. For example, if a surgeon knows that they are aiming for a further millimetre to be removed from the medial side of a resection surface and 2 mm from the posteromedial aspect of the resected surface. This can be well assessed by the surgeon by looking at the amount of bone taken on the recut. For example, if no solid piece of bone is available for further scanning then the bone removed will correspond to the predetermined kerf dimension of the saw blade. It is also envisaged that any additional solid pieces of bone that are subsequently resected from the joint or bone can be assessed for their exact dimensions and further modifications or updates to the model of residual bone made based on these dimensions.

Suitably, the methods described herein may further include the initial step of resecting the one or more resected portions from the joint or bone. Such resections may be carried out with a reciprocating bone saw or the like and cutting guides or blocks as are known in the art.

In one particular example, the methods described herein include the initial step of making a guide or preliminary resection or cut in the patient's joint or bone. The resected portions generated by the preliminary resection may then be utilised to generate a model of residual bone that allows for the accurate planning and positioning of cutting guides on the patient's bone or joint for making subsequent resections thereof.

Accordingly, in one form the present disclosure provides a method of performing surgery on a joint or bone of a patient, including the steps of:

(a) comparing a model of one or more resected portions resected from the joint or bone by a preliminary resection with a model of the patient's joint or bone prior to surgery; and

(b) determining a course of action associated with surgery on the joint or bone based at least partly on the comparison in (a).

With respect to step (a), this may include generating a model of residual bone of the patient's joint or bone, such as hereinbefore described.

In particular examples, the course of action can include determining one or more further resection planes for the joint or bone, such as those described herein. For such examples, the method may further include the step of further resecting the joint or bone based at least partly on the one or more further resection planes.

In some examples, the course of action can include determining a positioning of one or more cutting guides on the joint or bone to make one or more further resections therein. For such examples, the method may further include the step of further resecting the joint or bone based at least partly on the positioning of the one or more cutting guides thereon.

In certain examples, the method further includes the step of generating a model of one or more further resected portions that are produced by the further resections in the patient's joint or bone. As will be appreciated, the further resections to the patient's joint or bone will generate further resected portions which may be scanned, such as by those methods hereinbefore described, to generate a model thereof.

Suitably, the present method further includes the steps of:

(c) comparing the model of the further resected portions with a model of one or more implants, the model of the patient's joint or bone prior to surgery and/or the model of residual bone; and

(d) determining or selecting an implant for implantation into a patient's joint or bone based at least partly on the comparison in (c).

In this regard, step (c) of the present method may include the further step of generating a further model of residual bone based on such a comparison. The further model of residual bone may be generated as per previously described herein. Step (c) may further include comparing the further model of residual bone with the model of one or more implants, such as per those methods hereinbefore described.

Step (d) of the present method may be conducted as per those methods for selecting an implant described herein.

The preliminary resection is suitably of smaller or shorter dimensions than a final or finalised resection or cut in the joint or bone in question, such that relatively smaller resected portions are removed therefrom. Additionally, the preliminary resection can be made in an area of the bone or joint that allows for relatively easy access and visualisation and minimises saw blade variation or deviation from the required resection plane (i.e., a shorter length of resection can minimise saw blade deviation from the resection slot of the associated resection or cutting guide whilst making said resection). Furthermore, the preliminary resection suitably leaves sufficient bone in the joint for the engagement of cutting or resection guides thereon for making one or more further resections. Notwithstanding the above, the one or more resected portions generated from the preliminary resection are suitably of appropriate dimensions to be scanned such that a model thereof may be subsequently generated.

This model of the one or more resected portions generated from the preliminary resection may be utilised as described above to generate a model of residual bone. The model of residual bone may then function as a guide or landmark for the remaining further resections to be made to the joint or bone. In this regard, the preliminary resection allows for the development of a known position, reference point or landmark on the model of residual bone that advantageously allows for the accurate planning and positioning of cutting guides for the remaining further resections to be made to the patient's bone or joint.

By way of example, a preliminary resection in the distal femur may be made part way between an anterior and distal end of the femur and parallel to a finalised or final anterior chamfer resection plane in the femur (i.e., a shallow or shortened anterior chamfer resection). The skilled person will appreciate that this part of the femur is easily accessible during surgery and the resection length is short, thereby minimising any saw blade deviation during resection. The one or more resected portions removed from the anteriodistal region of the femur by the preliminary resection may then be scanned and a model of the resected portions generated therefrom. This model of the resected portions of the anteriodistal femur can then be utilised to generate a model of residual bone of the femur from a model of the patient's intact knee joint or femur, as previously described herein. The model of residual bone based on this preliminary resection facilitates the planning and positioning of cutting guides on the remaining bone of the femur and hence determining finalised or final versions of an anterior femoral resection plane, an anterior chamfer resection plane, a distal femoral resection plane, a posterior chamfer resection plane and/or a posterior femoral resection plane. Additionally, rotational aspects of these femoral resection planes can be determined from the model of residual bone.

Whilst the above embodiment is illustrated in respect of the anterior chamfer resection and the anteriodistal region of the femur, it is envisaged that preliminary resections in other regions of the femur (e.g., distal, posterior and posterior chamfer resection planes) could be utilised in a similar manner to that described above.

A similar method of resecting the proximal tibia may also be employed with a preliminary resection made part way between a proximal end of the tibia and a finalised or final proximal tibial resection (i.e., a shallow proximal tibial resection). A finalised or final proximal tibial resection plane may then be determined similar to that described about for the distal femur.

Once the further resections have been made in the patient's joint or bone, the further resected portions produced as a result may then be utilised as hereinbefore described to assist in determining an appropriate implant size for the patient's joint or bone. In this regard, a model of the further resected portions may be generated, such as by a scanning device described herein, which can then be compared to a model of one or more implants, the model of the patient's joint or bone prior to surgery and/or the model of residual bone. To this end, the model of the further resected portions may be utilised to generate an updated or further model of residual bone. The further resected portions and/or the further model of residual bone of the patient may then be compared in a similar manner to that described above with a range of implant models of known shape, size and/or type by, for example, software that then determines or selects the implant most appropriate for the patient's joint based on this comparison.

It is further envisaged that the methods described herein may include or allow for a determination of the laxity or tension present within the soft tissues surrounding the joint in question. By way of example, when bone resections have taken place (some or all) the bone surfaces can be distracted by one or more of the various tensors or spacer blocks known in the art. Such measurements can provide a total gap in the model of the patient's joint or bone from which the resected bone dimensions can be subtracted or the model of residual bone, which can result in a residual or resection space that corresponds to movement and/or laxity of the joint's soft tissues. This information can then be incorporated into the present methods and surgical decision making resulting therefrom.

In another aspect, the present disclosure provides, in part, a method of performing surgery on a bone of a patient, including the steps of:

(a) comparing a model of one or more resected portions resected from the bone with a model of one or more implants and/or a model of the bone prior to surgery; and

(b) determining a course of action associated with surgery on the bone based at least partly on the comparison in (a).

In certain examples, step (a) of the present method includes comparing the model of the one or more resected portions resected from the bone with the model of one or more implants and the model of the bone prior to surgery.

In some examples, step (a) of the present method includes comparing the model of the one or more resected portions resected from the bone with the model of one or more implants.

In other examples, step (a) of the present method includes comparing the model of the one or more resected portions resected from the bone with the model of the bone prior to surgery.

For the present aspect, the method or processes described herein may be readily applicable to osteotomy surgery, such as in respect of any bone in the body. In this regard, a surgeon could create a 3D model of one or more resected portions resected from a bone in question for closing wedge procedures and compare this to a model of the bone prior to surgery to determine if the resected resected portions are sufficient to achieve correct realignment of the bone. Furthermore, for opening wedge osteotomy procedures, a 3D model of one or more resected portions resected from the bone in question may be generated and then subtracted from a model of the bone prior to surgery. Alternatively, a model of residual bone may be generated directed by, for example, scanning the resected bone with a scanning device. A model of an implant to be inserted into the resected bone (e.g., bone cement, bone graft or other material inserted into the gap of the resected bone and then removed and scanned) may then be compared with a model of residual bone to determine whether or not the implant is sufficient to achieve optimal or correct realignment of the bone following implantation therein.

Steps (a) and (b) of the present method may be performed as previously described.

In view of the foregoing, and in a related aspect, the present disclosure relates to a method of determining or selecting an implant for implantation into a patient's joint or bone, said method including the steps of:

(a) comparing a model of one or more resected portions resected from the joint or bone with a model of one or more implants and/or a model of the joint or bone prior to surgery; and

(b) determining the implant for implantation into a patient's joint or bone based at least partly on the comparison in (a).

Steps (a) and (b) of the present method may be performed as previously described.

In a further aspect, the present disclosure provides a method of demonstrating the severity of a disease, disorder or condition in a patient's joint to the patient, said method including the steps of:

(a) generating a model of one or more resected portions resected from the joint; and

(b) providing the model to the patient.

In this regard, the bone resections can be analysed and a model, such as a 3D model thereof, provided for patient education to demonstrate their own personal disease severity. To this end, the model can clearly demonstrate specific detail of the patient's diseased joint or joint surface. This will provide meaningful education and insight for the patient and answering the common question or concern from patients as to the severity of the joint disease and whether surgery was indeed required. For the present method, the model of the one or more resected bone portions can also be compared to a model of the implanted prosthesis further demonstrating to patients how their pathology has been addressed.

The term “joint disease, disorder or condition”, as used herein, refers to an abnormal condition of the joints, in particular those due to injurious, traumatic, degenerative, inflammatory, infectious or autoimmune causes. Joint diseases, disorders or conditions include but are not limited to cartilage dysplasia, bone dysplasia, osteoporosis, osteoarthritis, rheumatoid arthritis, arthritis, synovitis, metabolic arthropathy, or joint disorders due to sports. The methods of the present disclosure can be used particularly for osteoarthritis.

Suitably, the model of the one or more resected portions is generated at least in part by a scanning device, such as that hereinbefore described.

Suitably, the model is provided to the patient by way of a portable storage medium as are known in the art, such as a CD, DVD or USB flash drive. In other embodiments, the model is provided to the patient in a solid or physical form, such as a printed 3D model.

In another aspect, the present disclosure provides an apparatus or system for assisting a surgeon performing surgery on a joint of a patient, the apparatus or system comprising a processor configured for comparing a model of one or more resected portions resected from the joint with a model of one or more implants and/or a model of the patient's joint prior to surgery and optionally determining a course of action associated with surgery on the joint based at least partly on the comparison.

In a related aspect, the present disclosure relates to an apparatus or system for determining or selecting an implant for implantation into a patient's joint or bone, the apparatus or system comprising a processor configured for comparing a model of one or more resected portions resected from the joint or bone with a model of one or more implants and/or a model of the joint or bone prior to surgery, and optionally determining or selecting the implant for implantation into a patient's joint or bone based at least partly on said comparison.

Suitably, the apparatus of the present aspect is for use in the method of any one of the aforementioned aspects.

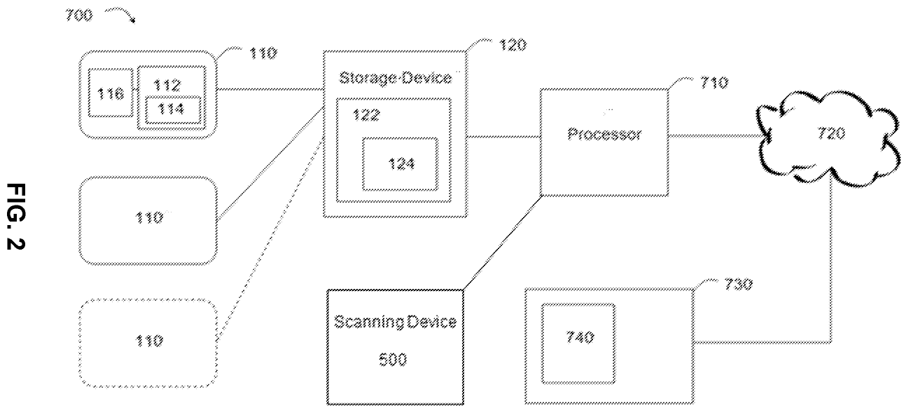

FIG. 2 illustrates an apparatus or system 700 according to one embodiment of the present disclosure. The apparatus 700 comprises a processor 710 in communication with one or more input devices 110 and a storage device 120. The processor 710 generates one or more reports 740 based on user input of the model of one or more bone portions resected from the joint, the model of one or more implants and/or the model of the patient's joint prior to surgery, entered via the input device 110. In alternative embodiments, the processor 710 is further configured to automatically generate from, for example, patient-specific anatomical data (e.g., a CT or MRI scan) the model of the particular joint in question, which may be received by the input device 110. Additionally, the processor 710 may be operably connected to a scanning device 500, such as via the input device, so as to directly receive the model of the one or more resected bone portions therefrom or alternatively receive scan data therefrom. In this regard, the processor 710 can be adapted to generate the model of the one or more resected bone portions based on the scan data received from the scanning device 500. Additionally, the processor 710 can be adapted to generate a model of residual bone of the joint in question based at least in part on the model of the one or more resected bone portions and the model of the patient's joint prior to surgery. In some embodiments, the processor 710 may further receive a measure of soft tissue tension of the joint by way of the input device 110 which may further facilitate determining course of action with respect to surgery of the patient's joint or bone.

Based on the above, the processor 710 can be further adapted to conduct comparisons of the model of the one or more resected bone portions with the model of one or more implants and/or the model of the patient's joint prior to surgery, as required by a user. The processor 710 can, for example, form part of a server which comprises the storage device 120 or be a separate computing device that is in communication with the storage device 120.

In particular embodiments, the processor 710 forms part of a computer, such as be a personal computer (PC), a tablet PC, a set-top box (STB), a Personal Digital Assistant (PDA), a cellular telephone, a web appliance, a network router, switch or bridge, or any computer capable of executing a set of instructions (sequential or otherwise) that specify actions to be taken by that computer, as are known in the art. The term “computer” shall also be taken to include any collection of computers that individually or jointly execute a set (or multiple sets) of instructions to perform any one or more of the methodologies discussed herein. The computer can operate as a standalone device or may be connected (e.g. networked) to other computers. In a networked deployment, the computer may operate in the capacity of a server, as described earlier, or a client computer in a server-client network environment, or as a peer computer in a peer-to-peer (or distributed) network environment.

The processor 710 provides a graphical user interface (GUI) 730 comprising the one or more reports 740 via a communications network 720, for example, to a computing device of a user or administrator. The one or more reports can include one or more metrics or readouts for, for example, determining: whether further resection of one or more resected surfaces or bones of the joint are required based on the comparison of the model of the resected bone portions and the model of the patient's joint prior to surgery; and, a particular implant type and/or size to be implanted into the joint in question based on the comparison of the model of the resected bone portions and the model of the one or more implants. In some embodiments, the one or more reports include one or more visualisations, classifications or models, such as those hereinbefore described, generated based on, for example, patient-specific anatomical data and scan data, and the GUI 730 can comprise one or more controls to select the one or more visualisations to be displayed.

The storage device 120 can comprise a computer memory 122 which can be, for example, a computer readable medium (e.g., software embodying or utilised by any one or more of the methodologies or functions described herein), such as, one or more hard disk drives or solid state drives. The computer memory 122 stores the patient-specific anatomical data, the scan data and the models. The computer memory 122 can also comprise computer readable code components 124 that when selectively executed by the processor 710 implements one or more aspects of the present disclosure, such as, generating aspects of the GUI 730 and providing the GUI 730 via the communications network 720.

Each input device 110 can comprise a computer memory 112 which can be, for example, a computer readable medium. The computer memory 112 comprises computer readable code components 114 (e.g., software embodying or utilised by any one or more of the methodologies or functions described herein) that when selectively executed by a processor 116 implements one or more aspects of the present disclosure, such as, generating and displaying the GUI 730 and receiving inputs, such as patient-specific anatomical data, scan data and models of the joint, the resected bones, and the implants, via the input device 110. In some embodiments, the computer memory 112 stores the patient-specific anatomical data, scan data or the models at the input device 110 prior to transmitting the data to the storage device 130. In further embodiments, the storage device 130 stores a plurality of models of known implants from a range of commercial providers as are known in the art. The computer readable code components 114 may further be transmitted or received over a network via the communications network 720 utilising any one of a number of well-known transfer protocols (e.g., HTTP, UDP, TCP, USSD, FTP).

In one further aspect, the present disclosure describes a computer-readable medium, such as a non-transitory computer-readable medium, having stored thereon a computer program, which, when executed by a computer, causes the computer to perform the method of any one of the aforementioned aspects.

As used herein, the terms “approximately” and “about” refer to tolerances or variances associated with numerical values recited herein. The extent of such tolerances and variances are well understood by persons skilled in the art. Typically, such tolerances and variances do not compromise the structure, function and/or implementation of the devices and methods described herein.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

1. A method of performing surgery on a joint or bone of a patient, including the steps of:
(a) comparing a model of one or more resected portions resected from the joint or bone with a model of one or more implants and/or a model of the patient's joint or bone prior to surgery; and
(b) determining a course of action associated with surgery on the joint or bone based at least partly on the comparison in (a).

2. The method of claim 1, wherein the model of the one or more resected portions, the model of the one or more implants and/or the model of the patient's joint or bone are or comprise respective three dimensional (3D) models.

3. The method of claim 1, further including the initial step of:
(a) generating the model of the one or more resected portions resected from the joint or bone; and/or
(b) generating the model of the patient's joint or bone prior to surgery.

4. The method of claim 3, wherein the model of the one or more resected portions resected from the joint or bone is generated at least in part by a scanning device.

5-6. (canceled)

7. The method of claim 36, wherein the model of the patient's joint or bone is generated at least in part by radiological imaging, magnetic resonance imaging (MRI) and/or computed tomography (CT).

8. The method of claim 1, wherein step (a) comprises overlaying the model of the one or more resected portions resected from the joint or bone with the model of the patient's joint or bone.

9. The method of claim 8, further comprising subtracting the model of the one or more resected portions resected from the joint or the bone from the model of the patient's joint or bone to generate the model of residual bone thereof.

10. The method of claim 1, including the step of comparing the model of the one or more implants and/or the model of the patient's joint or bone with the model of residual bone.

11. The method of claim 1, wherein the course of action comprises:
(a) further resection of one or more resected surfaces or bones of the joint or the bone; and/or
(b) determining an implant size for the patient's joint or bone based on the comparison in (a).

12. The method of claim 11, wherein further resection is the course of action when the comparison of: (i) the model of the one or more resected portions and/or the model of residual bone with the model of the patient's joint or bone; and/or (ii) the model of the one or more implants with the model of residual bone; indicates insufficient and/or improper resection of one or more bones of the joint or the bone.

13. (canceled)

14. The method of claim 1, wherein the course of action includes determining an implant size for the patient's joint or bone based on the comparison in (a).

15-16. (canceled)

17. The method of claim 1, further including the initial step of resecting the one or more resected portions from the joint or bone.

18. The method of claim 1, further including the initial step of making a preliminary resection in the joint or bone so as to produce the one or more resected portions.

19. The method of claim 18, wherein the course of action comprises: (i) determining one or more further resection planes for the joint or bone based at least partly on the preliminary resection; and (ii) optionally further resecting the joint or bone based at least partly on the one or more further resection planes.

20. The method of claim 1, wherein the joint is suitably a knee joint, a shoulder joint, an ankle joint or a hip joint.

21-35. (canceled)

36. A system for assisting a surgeon in performing surgery on a joint of a patient, the system comprising a processor configured for: (a) comparing a model of one or more resected portions resected from the joint with a model of one or more implants and/or a model of the patient's joint prior to surgery; and (b) optionally determining a course of action associated with surgery on the patient's joint based at least partly on the comparison in (a).

37. A system for determining or selecting an implant for implantation into a patient's joint or bone, the apparatus or system comprising a processor configured for:
(a) comparing a model of one or more resected portions resected from the joint or bone with a model of one or more implants and/or a model of the joint or bone prior to surgery; and
(b) optionally determining or selecting the implant for implantation into a patient's joint or bone based at least partly on the comparison in (a).

38. The system of claim 36, further comprising a scanning device for generating the model of the one or more resected portions resected from the joint.

39. (canceled)

40. A computer-readable medium having stored thereon a computer program, which, when executed by a computer, causes the computer to perform a method, including the steps of:
(a) comparing a model of one or more resected portions resected from the joint or bone with a model of one or more implants and/or a model of the patient's joint or bone prior to surgery; and
(b) determining a course of action associated with surgery on the joint or bone based at least partly on the comparison in (a).

41. The system of claim 37, further comprising a scanning device for generating the model of the one or more resected portions resected from the joint.
